(19) United States
(12) Patent Application Publication
(10) Pub. No.: US 2026/0301964 A1
(43) Pub. Date: Oct. 1, 2026

(54) MULTIMODAL ARTIFICIAL NEURAL NETWORK METHOD AND SYSTEM FOR PRECISION ONCOLOGY

(71) Applicants: OneCell Diagnostics, Inc., Foster City, CA (US); OneCell Diagnostics India Pvt. Ltd., Pimpri-Chinchwad (IN)

(72) Inventors: Jayendra Shinde, Mumbai (IN); Anand Raj Ulle, Mysuru (IN); Chongtham Cha Chinglemba, Imphal West (IN); Thoudam Yoihenba, Imphal West (IN); Gowhar Shafi, Mumbai (IN); Mohan Uttarwar, Cupertino, CA (US)

(21) Appl. No.: 19/413,828

(22) Filed: Dec. 9, 2025

(30) Foreign Application Priority Data
Mar. 29, 2025 (IN) 202521030746

(51) Int. Cl. G16H 50/30; G06V 10/774; G06V 10/82; G06V 20/69; G16B 25/10; G16B 40/20

(52) U.S. Cl. CPC G16H 50/30; G06V 10/774; G06V 10/82; G06V 20/695; G16B 25/10; G16B 40/20; G06V 2201/03

(57) ABSTRACT

A novel multi-modal artificial neural network-based method and system for predicting patient response and biomarkers for precision oncology from histopathological whole slide images (WSIs) only. Utilizing an artificial neural network (ANN) with a feature extraction module, image to gene expression prediction, encoders and contrastive learning-based methods the system processes WSIs to determine treatment scores from predicted profiles, forecasting responses to therapies like chemotherapy, targeted drugs, and immunotherapy across cancers (e.g., breast). Applications include predicting recurrence risk, treatment efficacy (e.g., PARP inhibitors, checkpoint inhibitors), biomarker status, patient stratification, clinical trial design, drug target identification, and therapeutic combination efficiency.

### CROSS-REFERENCE TO RELATED APPLICATIONS

This application claims priority to co-pending Indian Patent Application No. 202521030746, filed on Mar. 29, 2025, which application is incorporated by reference in its entirety herein.

### FIELD

The present invention relates generally to the field of artificial intelligence and machine learning in oncology, and more particularly to systems and methods for predicting patient response to tasks related to precision oncology using multi-modal data integration and analysis, with a specific emphasis on histopathological image analysis.

### BACKGROUND

Histopathology remains the gold standard for cancer diagnosis and prognosis, relying on stained tissue slides, such as those with hematoxylin and eosin (H&E), to assess tumor morphology (Bostwick et al., 2008; Elmore et al., 2015). However, molecular insights from genomic profiling are increasingly critical for personalized cancer treatment (Cancer Genome Atlas Research Network, 2013; Dienstmann et al., 2017). These tests typically require genomic sequencing or specialized assays, which are costly, time-intensive, and not universally accessible (Collins & Varmus, 2015).

Histopathological images, generated from tissue biopsies, contain a wealth of morphological and biological information that is often underutilized for predictive purposes (Madabhushi & Lee, 2016). Digital histopathology, combined with AI, offers a promising alternative by extracting molecular insights directly from readily available histopathology images (Coudray et al., 2018; Lu et al., 2021).

Prior approaches have used machine learning to analyze whole slide images (WSIs) for tasks like tumor classification, mutation detection, and survival prediction (Campanella et al., 2019; Kather et al., 2019). However, these methods often focus on direct phenotypic predictions and lack the ability to infer comprehensive genomic profiles or predict responses to specific cancer therapies without extensive treatment-specific datasets (Mobadersany et al., 2018; Yamashita et al., 2021).

There exists a need for a cost-effective, rapid, and generalizable method to predict patient-specific responses to cancer therapies using histopathology images alone, bridging the gap between morphological and molecular data.

### SUMMARY

It has now been surprisingly found that various tasks related to precision oncology can be addressed by examining and analyzing only the histopathological image of a cancer patient using a multimodal artificial neural network-based method and system disclosed herein.

According to an embodiment is disclosed a multimodal artificial neural network (ANN) based method and system for precision oncology.

According to an embodiment is disclosed a method for performing a task related to precision oncology comprising: extracting the morphological and architectural features from the standard hematoxylin and eosin stained whole slide image (WSI) of the biopsy tissue of the patient representative of the said task, extracting the features relevant to the said task and transferring the said information to a trained (Transcriptome-guided Representation Inference Insights for Tumor Yield) TRINITY AI model and process and generate the output.

According to the embodiment of the disclosure, for a given precision oncology task, a final prediction report along with a relevant biomarker score indicating the predicted refined gene expression profile of subset of genes is generated.

According to an embodiment of the disclosure, the aspects of precision oncology tasks include but are not limited to predicting response to therapeutic interventions, selecting the most effective therapy for a patient, treating patients based on selected intervention, ranking patients for clinical trial eligibility, designing clinical trials for likely responders, and monitoring treatment response over time.

According to an embodiment of the disclosure, the method predicts the outcomes of the precision oncology tasks based on histopathology images only

According to an embodiment of the disclosure, the artificial intelligence network model is developed and trained to process and integrate the following types of data:

Histopathological whole slide images (WSI) obtained from standard pathology practice,

Bulk RNA-seq data (primarily for training the TRANSCRIPT - Transcriptomics-guided Representation Analysis and Normalization for Slide-based Computation for Integrated Pathology Tasks model and process),

Clinical data, including but not limited to patient demographics, tumor characteristics, biomarker measurements, treatment history, comorbidities, family history, lifestyle factors, quantitative features extracted from pathology reports, and other relevant clinical information.

According to an embodiment of the disclosure, the Whole Slide Image (WSI) input is converted into relevant features using one or more feature extraction architectures, which may include convolutional neural networks, vision transformers, or other foundation models.

According to an embodiment of the disclosure, provided herein is TRANSCRIPT, an model and process for predicting gene expression profiles from histopathological images.

According to an embodiment of the disclosure, the TRANSCRIPT model and process is trained through a method comprising: (a) providing a training dataset of paired histopathological images and corresponding bulk RNA-seq data from the same tissue samples; (b) preprocessing the histopathological images through normalization, quality control, and patch extraction; (c) preprocessing the RNA-seq data through normalization and batch correction; (d) training the model and process to minimize the difference between predicted gene expression values and actual gene expression values measured by bulk RNA-seq.

According to an embodiment of the disclosure, the predicted gene expression values and actual gene expression values correlation was measured to be between Pearson Correlation Coefficient (PCC) 0.25 to 0.95.

According to an embodiment of the disclosure, TRANSCRIPT generates predicted gene expression levels for a predetermined set of genes.

According to an embodiment of the disclosure, the TRANSCRIPT model and process is validated using a separate validation dataset to assess generalization performance and prevent overfitting.

According to an embodiment of the disclosure, the TRANSCRIPT model and process is fine-tuned to optimize performance on clinically relevant gene sets.

According to an embodiment of the disclosure, the TRANSCRIPT model and process can be used to gain molecular insights without requiring additional tissue sampling or next generation sequencing based genomic testing.

According to an embodiment of the disclosure, TRANSCRIPT model and process outputs gene expression profiles from histopathological images, thereby providing input to SCOUT (Selecting Candidates Over/Under –expressed Transcripts) model and process.

According to an embodiment of the disclosure, SCOUT, a computational method for gene selection and dimensionality reduction, comprises providing a gene selection system configured to a) receive a comprehensive gene expression profile comprising thousands of predicted genes from the TRANSCRIPT model and process; b) implement multiple selection approaches including differential gene expression analysis and genetic model and process-based selection; c) identify a reduced subset of genes with optimal predictive value for specific clinical outcomes; d)output a refined gene expression profile containing approximately subset of genes with heightened relevance to the target condition.

According to an embodiment of the disclosure, SCOUT performs a gene selection process guided by precision oncology tasks to ensure selected genes have meaningful biological and clinical significance.

According to an embodiment of the disclosure, the refined gene expression profile from SCOUT serves as direct input to the Gene Expression Encoder model and process of the TRINITY (Transcriptome-guided Representation Inference Insights for Tumor Yield) AI model and process, enhancing computational efficiency and improving signal-to-noise ratio for downstream predictive tasks.

According to an embodiment of the disclosure, the gene expression encoder model and process is configured to receive refined gene expression data from the SCOUT model and process and produce a gene expression embedding vector of predetermined dimensionality through a series of transformations designed to capture relevant biological patterns.

According to an embodiment of the disclosure, the WSI encoder model and process is configured to receive a histopathological whole slide image features and produce a WSI embedding vector of predetermined dimensionality. This model and process may utilize various deep learning architectures including convolutional neural networks (CNNs), ResNet-based models, or vision transformers, and may incorporate attention-based approaches for processing multiple regions of interest within the image.

According to an embodiment of the disclosure, the clinical data encoder model and process is configured to receive structured clinical data and produce a clinical data embedding vector of predetermined dimensionality, employing appropriate encodings for different data types and handling missing values through suitable computational methods.

According to an embodiment of the disclosure, TRINITY AI is a machine learning model (such as a contrastive learning-based model and process) that aligns the embedding vectors from the three modalities as described above, in a shared representation space using a contrastive loss function. This loss function enables the model and process to learn meaningful representations by maximizing agreement between different views of the same subject while distinguishing between different subjects.

According to an embodiment of the disclosure, this model and process is the trained TRINITY AI model and process.

According to an embodiment of the disclosure, TRINITY AI prediction layer receives the aligned embedding vectors and generates output for relevant precision oncology related tasks. This model and process may implement various prediction approaches suitable for different precision oncology tasks with accuracy ranging between 80-98%.

According to an embodiment of the disclosure, a system and method is provided for predicting breast cancer recurrence risk that yields prognostic information substantially equivalent to that of the commercially available genomic assays without necessitating additional tissue sampling or RNA sequencing procedures. The aforementioned commercially available genomic tests comprise an analysis of expression levels of predetermined genes to generate a test score to stratify patients according to predetermined ranges for the purpose of guiding adjuvant chemotherapy decisions in patients diagnosed with early-stage, hormone receptor-positive breast cancer. Said genomic tests requires specialized tissue preparation protocols, and substantial laboratory processing time, thereby resulting in increased healthcare expenditures and treatment initiation delays.

According to an embodiment of the disclosure, the method as described herein, provides alternative methodology for predicting breast cancer recurrence risk utilizing exclusively standard histopathological whole slide images (WSI) that are routinely collected in standard clinical practice.

According to an embodiment of the disclosure, the method as described herein, provides alternative methodology for addressing precision oncology tasks and processing.

According to an embodiment of the disclosure, the method comprises: processing said WSIs to extract relevant morphological and textural features indicative of recurrence risk; generating WSI embeddings through implementation of specialized neural network architectures; and producing a recurrence risk score via trained TRINITY AI model and process, whereby patients are classified into appropriate risk categories for subsequent treatment planning.

According to an embodiment of the disclosure, the validation studies conducted across multiple independent cohorts, TRINITY AI demonstrated robust performance metrics in the independent external validation cohort (n= 166) with 64.5% sensitivity 93.3% specificity, positive predictive value of 69.0% and negative predictive value of 92.0%. This level of predictive accuracy enables healthcare practitioners to make informed treatment decisions substantially comparable to those guided by genomic testing methodologies, while potentially reducing healthcare expenditures, and expediting treatment planning timelines.

According to an embodiment of this disclosure, the patient sample for analysis is selected from; biopsy samples containing cancer cells, core needle biopsy samples containing cancer cells taken using fine needle aspirates.

According to an embodiment of this disclosure, the samples are formalin fixed and paraffin embedded (FFPE).

According to an embodiment of this disclosure, a thin slice of the tissue from the FFPE block is stained with hematoxylin and eosin stains over a glass slide.

According to an embodiment of this disclosure, a WSI image is created from the above slide using a whole slide scanner.

According to an embodiment of the disclosure, the preprocessing module, comprises a quality check, which is an automated program, comprising set of instructions, to read, process and output the target regions of interest from the digitized H and E-stained whole slide image, stored on a computer readable medium.

According to an embodiment of the disclosure, the QC extracts the real tissue regions of interest by excluding the artefacts, staining/sectioning errors, blurry and unwanted biological substances such as fatty tissue, small objects, and white background fatty regions.

According to an embodiment of the disclosure, the processor has a) a dual microprocessor b) multi-processor architectures.

According to an embodiment of the disclosure, the processor is a CPU (Central processing unit).

According to an embodiment of the disclosure, the processor is a CPU and a GPU (Graphic processing unit).

According to an embodiment of the disclosure, the processor is a CPU and a TPU (Tensor processing unit).

According to an embodiment of the disclosure, image acquisition system comprises a whole slide digital scanner, connected to a processor and a computer readable medium to acquire the digitized formats of the whole glass slides of the H and E-stained tissue samples and store them into an image data format in a computer readable medium.

According to an embodiment, this disclosure provides a system that comprises a scanner that creates whole slide image from the tissue biopsy samples containing cancer cells, core needle biopsy samples containing cancer cells taken using fine needle aspirates as well as samples obtained by using relevant techniques.

According to an embodiment of the disclosure, the whole slide scanner supplied by Phillips, Hamamatsu, Aperio, etc. are suitable for the purpose.

According to an embodiment of the disclosure, digitized H and E-stained whole slide image is represented in a pyramidal structure of different zoom levels, representing the slide at different zoom levels.

According to an embodiment of the disclosure, the various parts of the image acquisition apparatus are connected over internet, which comprises of communication networks such as WAN / LAN, devices such as gateways / routers / switches / bridges and communication protocols such as TCP/IP. The appended claims may serve as a summary of this application.

### BRIEF DESCRIPTION OF THE DRAWINGS

FIG. 1 is a diagram illustrating a Training TRINITY AI model and process in accordance with the present invention.

FIG. 2 is a diagram illustrating a Training TRANSCRIPT model and process in accordance with the present invention.

FIG. 3 is a diagram illustrating a SCOUT model and process in accordance with the present invention.

FIG. 4 is a diagram illustrating a TRINITY AI prediction output in accordance with the present invention.

FIG. 5 is a diagram illustrating a comprehensive expansion of the WSI processing under the Quality Control processing.

FIG. 6 is a diagram illustrating a visualization of TRINITY AI's Learned Multi-Modal Embeddings by HR+/HER2-.

FIG. 7 is a diagram illustrating a UMAP Visualization of TRINITY AI Embeddings by recurrence risk scores.

FIG. 8 is a diagram illustrating a UMAP Visualization by MKI67 Expression.

FIG. 9 is a diagram illustrating a UMAP Visualization by ESR1 Expression.

FIG. 10 is a diagram illustrating a UMAP Visualization by PGR Expression.

FIG. 11 is a diagram illustrating a UMAP Visualization by ERBB2 Expression.

FIG. 12 is a diagram illustrating an exemplary process 1200 that may perform processing in some embodiments.

FIG. 13 is a diagram illustrating a illustrating an exemplary computer that may perform processing in some embodiments.

### DETAILED DESCRIPTION OF THE DRAWINGS

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and their equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well-known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments are implemented by a computer system. A computer system may include a processor, a memory, and a non-transitory computer-readable medium. The memory and non-transitory medium may store instructions for performing methods and steps described herein.

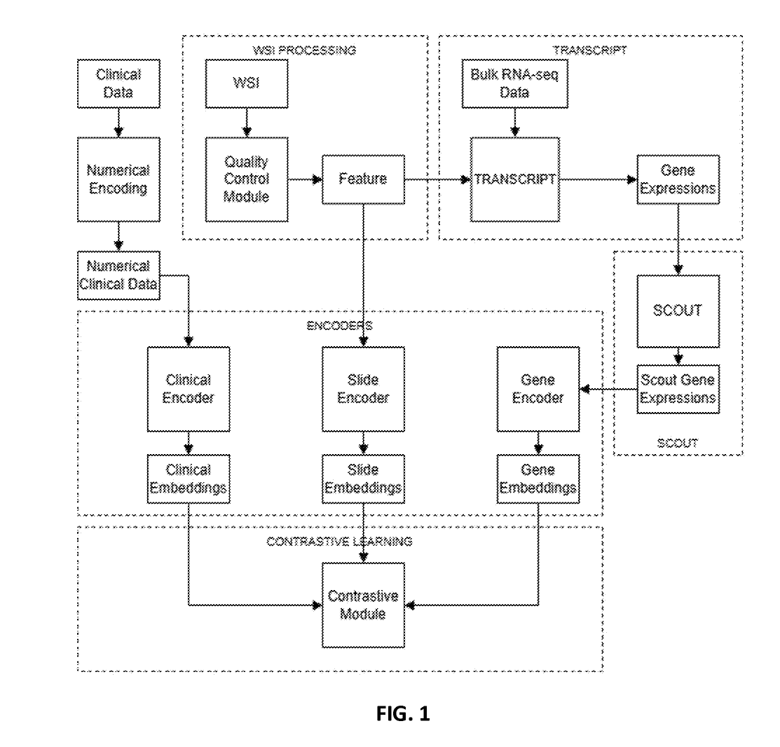

FIG. 1 illustrates a schematic block diagram of the TRINITY AI model and process, depicting the system architecture and data flow between interconnected computational model and process. The diagram comprises five main sections demarcated by dashed lines: WSI PROCESSING, TRANSCRIPT, SCOUT, ENCODERS, and CONTRASTIVE LEARNING.

The WSI PROCESSING section on the upper left shows the workflow for processing whole slide images (WSI), beginning with raw WSI input, which feeds into a Quality Control Module that also contains the Tile Extractor Module that segments the image into qualified discrete tiles. These tiles are then processed by a Feature Extractor Module to generate features representative of histopathological patterns.

The TRANSCRIPT section on the upper right illustrates the computational pipeline that processes Bulk RNA-seq Data as input to the TRANSCRIPT model and process, which generates Gene Expressions output. The Gene Expressions serve as input to both the SCOUT model and process and the encoder section.

The SCOUT section on the right portrays the gene selection and dimensionality reduction process, where Gene Expressions from the TRANSCRIPT model and process are refined by the SCOUT model and process to produce Scout Gene Expressions with enhanced predictive value.

The ENCODERS section in the middle of the diagram illustrates three parallel encoding pathways: (1) a Clinical Encoder processing Clinical Textual Data to generate Clinical Embeddings; (2) a Slide Encoder processing features from the WSI processing pipeline to generate Slide Embeddings; and (3) a Gene Encoder processing the Scout Gene Expressions to generate Gene Embeddings.

The CONTRASTIVE LEARNING section at the bottom depicts the Contrastive Module that receives and integrates the three types of embeddings (Clinical, Slide, and Gene) through a contrastive learning framework to create a unified representation for downstream predictive tasks.

The diagram illustrates the end-to-end multimodal data integration approach of the TRINITY AI model and process, showing how diverse cancer-related data types are processed, encoded, and aligned to enable comprehensive cancer prognosis prediction.

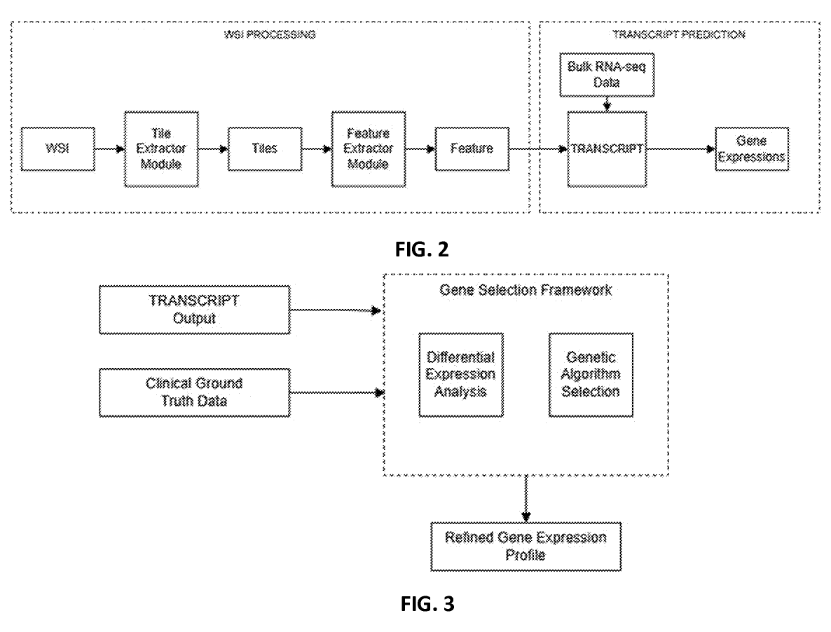

FIG. 2 illustrates a schematic block diagram of the TRANSCRIPT model and process of the TRINITY AI model and process, depicting the computational pipeline for predicting gene expression profiles from histopathological images. The diagram comprises two main sections demarcated by dashed lines: WSI PROCESSING and TRANSCRIPT PREDICTION.

The WSI PROCESSING section on the left shows the workflow for processing whole slide images (WSI), beginning with raw WSI input, which is directed to a Tile Extractor Module that segments the whole slide image into discrete tiles. These tiles then proceed to a Feature Extractor Module that generates histopathological features represented as a Feature output. This pipeline enables the extraction of relevant visual patterns and characteristics from complex histopathological images.

The TRANSCRIPT PREDICTION section on the right depicts the gene expression prediction process, where Bulk RNA-seq Data is shown as an input during the training phase of the TRANSCRIPT model and process. The TRANSCRIPT model and process receives the Feature output from the WSI processing pipeline and generates predicted Gene Expressions as output. This illustrates how the system converts histopathological image features into molecular-level information without requiring additional tissue sampling or genomic testing.

The diagram illustrates the end-to-end process by which the TRANSCRIPT model and process bridges the gap between histopathological imaging data and gene expression profiles, enabling the extraction of molecular insights directly from standard clinical histopathology slides.

FIG. 3 illustrates a schematic block diagram of the SCOUT model and process of the TRINITY AI model and process, depicting the gene selection and dimensionality reduction process. The diagram shows the workflow for refining gene expression profiles to identify the most predictive subset of genes for cancer prognosis.

On the left side of the diagram, two inputs are shown: "TRANSCRIPT Output" representing the comprehensive gene expression profiles generated by the TRANSCRIPT model and process, and "Clinical Ground Truth Data" representing patient outcome data and clinical information used to guide the gene selection process.

The central portion of the diagram contains a dashed box labelled "Gene Selection Framework" which encompasses two parallel selection approaches: "Differential Expression Analysis" and "Genetic Model and process Selection." These two complementary methodologies work in concert to identify genes with optimal predictive value for specific clinical outcomes.

The output of the Gene Selection Framework flows to the "Refined Gene Expression Profile" shown at the bottom of the diagram. This refined profile represents a reduced subset of approximately 500 genes with heightened relevance to the target condition, as indicated in the specification.

The diagram illustrates how the SCOUT model and process performs dimensionality reduction on gene expression data while preserving and enhancing the predictive signal relevant to cancer prognosis, thereby improving computational efficiency and signal-to-noise ratio for downstream predictive tasks in the TRINITY AI model and process.

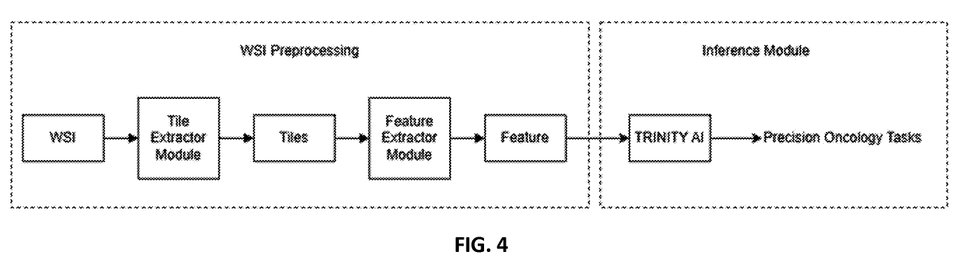

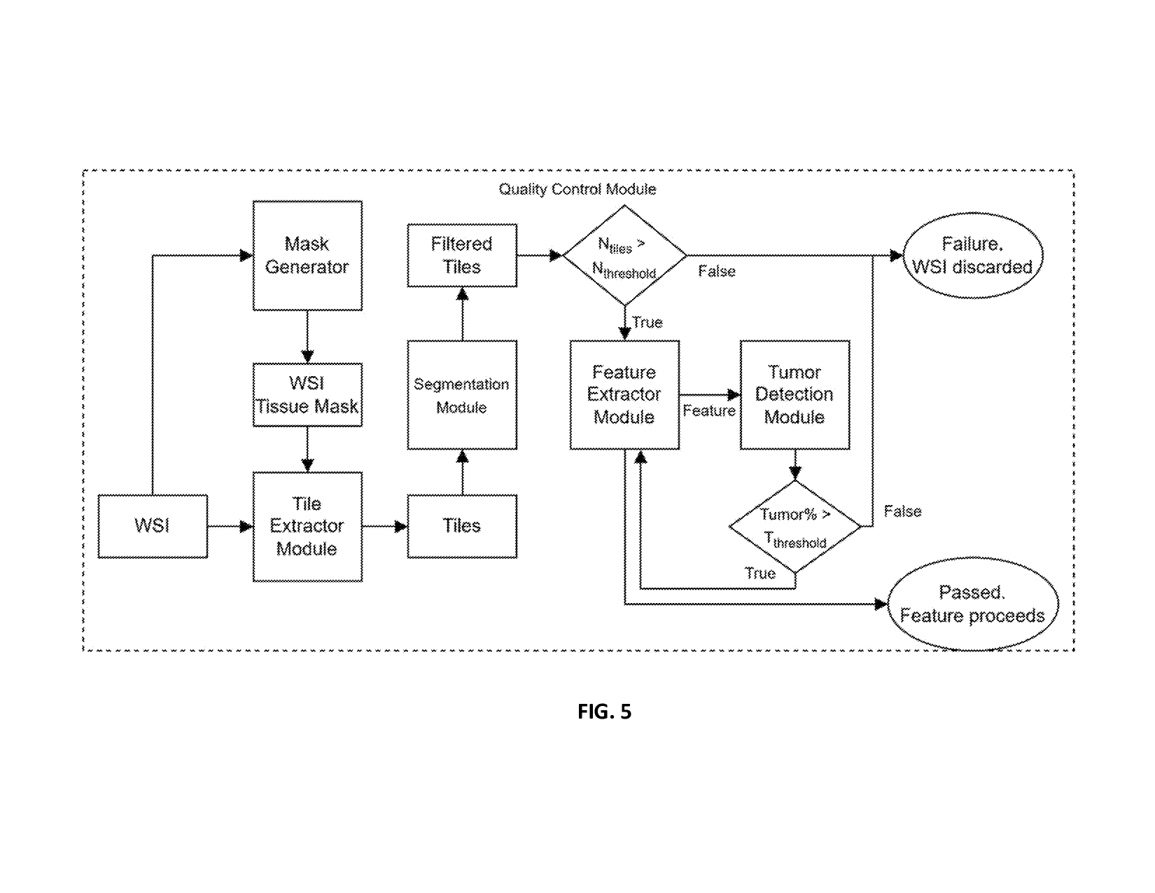

FIG. 4 illustrates aTRINITY AI prediction output pipeline consisting of two modules: the WSI Preprocessing and an Inference Module, designed to perform precision oncology tasks. In the preprocessing module, a WSI is processed by first tiling it and then encoding the morphological patterns by the feature extractor. In the Inference Module, the extracted features are processed by the TRINITY AI model and process, which performs downstream precision oncology tasks.

FIG. 5 describes the comprehensive expansion of the WSI processing under the Quality Control processing segment. This diagram shows the process creating and process of various data inputs and generation and processing of the vector embeddings.

In some embodiments, the WSI (Whole Slide Images) are processed with a quality control module. For example, the Whole Slide Image (WSI) undergoes an initial Quality Control (QC) routine, referred to as “Mask Generator (MG)”. The primary objective of this MG step is to ensure the selection of high-integrity tissue regions for downstream analysis. The WSI is input into the MG module, which produces a clean tissue mask. The clean tissue mask is a binary image, where the foreground regions (valid tissue) are typically white with a pixel value of 1 or 255 (in 8-bit format) and background regions (artifacts/unwanted regions) are black with a pixel value of 0. The mask is typically stored as an image file usually in PNG or TIFF format. The mask is a pixel-level annotation, where each pixel represents either inclusion of tissue regions or exclusion of non-tissue regions. Usually, the tissue regions are white as opposed to black in the non-tissue regions. The resolution of mask is usually lower than that of the WSI, typically 1.25x corresponding to a pixel size of 8-10 micrometre. After the WSI passes MG, it, along with the generated mask, is forwarded to the Tile Extraction Module for high resolution tile generation.. The "removal" of artifacts or irrelevant regions occurs in the binary mask, not the original WSI. The mask marks usable tissue as 1 (white) and everything else (artifacts/background) as 0 (black). To create this binary mask, irrelevant or artifact-heavy regions are identified from the downscaled image using classical computer vision techniques such as colour-based filtering, texture analysis, morphological analysis and sometimes even machine learning which can classify between valid tissue and other unwanted regions. These unwanted regions are in the “excluded” class. They exist in the original WSI, but the mask indicates to the analysis pipeline that they should be ignored.

The Tile Extraction Module is a parallelized model and process designed to meticulously extract tiles based on both physical and digital properties of the WSI:
Physical Properties include
   a. Microns per Pixel (MPP)
   b. Magnification level of the WSI
   c. Required patch micron size
Digital Properties include
   a. Patch pixel dimensions
   b. Stride parameters
   c. Downscaling factors, among others

The WSI is stored as multi-resolution image pyramids, enabling viewing at different magnifications without loading the full-resolution image. Typically, at 40x (high-resolution), the pixel size is around 0.25 microns per pixel and at 20x magnification, the pixel size is around 0.5 microns per pixel. For example, if a digitized slide is at 40x with 0.25 microns per pixel, and the tissue size is 20 x 15 mm, the resulting image size would be approximately 80,000 x 60,000 pixels. The mask is generated and saved at a specific pyramid level, whose down sampling factor is known (e.g., down sampling 32 times to generate a mask of 1.25x). By keeping track of this down sampling factor, each coordinate in the mask can be translated back to the coordinates at the full WSI resolution. This massively reduces pixel count, speeds up processing, and conserves memory and storage, while still preserving sufficient detail for differentiating tissue from background or artifacts. The image processing method described herein provides a technical solution to the technical problem of image processing. Additionally, this process improves the computer system by reduction of computation resources needed to process the images.

For tile extraction
a. The required pixel size per tile is computed using the MPP and the desired physical dimensions (patch micron size).
b. Using this computed size, valid tile coordinates are identified from the downscaled image.

A tile is validated if it contains ≥90% tissue coverage as per the mask.

Valid tile coordinates are utilized to extract the corresponding high-resolution tiles from the original WSI in parallel.

In addition to this structured and parameterized pipeline, an additional layer of quality refinement is performed using a deep learning-based segmentation model. This model is employed to evaluate each tile for the presence of:
a. Blur
b. Tissue fold
c. Background content

Each of these parameters is quantified, and tiles are discarded if they exceed predetermined thresholds for any of the above quality metrics. Furthermore, WSIs that yield an insufficient number of valid tiles—below a predefined minimum tile count threshold (1000 tiles)—are also excluded from subsequent processing, as they are deemed inadequate for robust analysis. Following this, a deep learning-based tumour detection model is applied to estimate tumour content across the retained clean tiles. WSIs that exhibit insufficient tumour presence (<10%), based on this prediction, are further excluded. This final step ensures that only clean, artifact-free, and tissue-relevant tiles are retained for downstream analysis.

As a result, each WSI yields a collection of high-quality tiles, precisely filtered and through learned segmentation criteria, preserving only the most diagnostically relevant regions.

In some embodiments, certain WSI features are generated. For example, each validated tile from the WSI is processed to generate high-dimensional feature vectors. Specifically, each tile is encoded into a 1536-dimensional numerical vector, where each dimension captures complex semantic and structural information inherent in the tissue morphology. These feature vectors are collectively referred to as WSI feature stacks and are designed to preserve critical diagnostic and prognostic patterns for subsequent modelling.

In some embodiments, the contrastive model is a learning framework composed of three parallel encoder networks, each corresponding to one of the three distinct data modalities: WSI, gene expression, and clinical data. Each encoder learns to generate fixed length embedding vectors (512 dimension) that inhabit a shared representation space.

The contrastive learning objective ensures the alignment and separation of these embeddings via the following mechanism:
a. Positive pairs: Vectors derived from the same sample across different modalities are encouraged to align closely within the shared space.
b. Negative pairs: Vectors not sharing sample identity are repelled from one another to maintain separation in the representation space.
c. Through this learning dynamic, the model encodes both intra-modal features and cross-modal relational knowledge. Each encoder can:
d. Ingest modality-specific data,
e. Extract latent semantic and biological characteristics,
f. Embed them in a harmonized vector space conducive for multi-modal fusion.

The encoders themselves may consist of standard neural networks, transformer-based architectures, or attention mechanisms, depending on implementation. The contrastive learning objective is achieved through InfoNCE loss function.

Vector embeddings are generated from the TRINITY AI model and process. Each trained encoder within the TRINITY AI architecture is capable of computing high-dimensional embedding vectors (512 dimension) that represent individual samples. These embeddings reflect complex biological, morphological, and clinical characteristics, encapsulated into a unified, learned projection space. The dimensionality of these vectors is consistent across modalities, facilitating integrated analysis. These embeddings can be further projected to lower dimensions (e.g., via UMAPs – refer below) for visualization, aiding in the interpretation of modality relationships and feature clustering.

The contrastively trained embeddings from TRINITY AI model and process are utilized in downstream processing. For example, post contrastive training, embeddings from any of the three modalities can be utilized as feature inputs for downstream predictive tasks. Specifically:
a. Embeddings are extracted using the corresponding trained encoder.
b. These embeddings are coupled with known sample labels (e.g., risk class, treatment response).
c. A secondary machine learning or deep learning model and process is then trained using these embeddings to perform specific tasks such as classification, regression, or survival analysis within precision oncology.
d. More specifically, the embeddings are trained against the recurrence risk classes using a machine learning model to predict the recurrence risk classes, which can also give the scores using the probability.

The system uses clinical data as part of its processing. The clinical data comprises a combination of textual, ordinal, continuous and categorical variables that capture patient-specific information relevant to the oncology task. These raw data types are numerically encoded in a manner that preserves the underlying distribution within each clinical variable, ensuring that no semantic or ordinal structure is lost during preprocessing. The resulting numerically encoded data serves as input to the clinical encoder, which learns a structured vector representing the clinical profile of the patient.

In some modalities, the contrastive framework is paired. All three modalities—WSI, gene expression, and clinical data—are paired at the sample level. This pairing ensures that embeddings learned from different modalities pertain to the same biological sample, which is essential for effective contrastive learning and cross-modal representation alignment.

In some embodiments, inferencing is performed on new sample. Inference is conducted using WSI modality. The inference process is as follows:

The WSI data undergoes pre-processing similar or identical to the training phase.

Corresponding features (e.g., 1536-dimensional WSI feature stacks) are computed.

These features are input into the pre-trained encoder to generate the embedding vector.

The resulting embedding is then passed into the downstream model (e.g., classifier or risk model) trained specifically on the WSI embeddings to produce predictions relevant to the oncology task at hand.

Apart from the resultant values, a predictive certainty score is also given out to enhance interpretability and transparency of the prediction. The certainty/confidence is calculated in the following way:

A diverse set of XGBoost models was trained with varying initialization seeds. Models demonstrating strong discriminative capability (training AUC > 0.95) were selected for further evaluation. This filtering ensures only well-performing models contribute to the ensemble predictions.

For each accepted model
a. Raw class probabilities were predicted on the held-out test set.
b. A decision threshold was determined to achieve 80% sensitivity, calibrated using an external validation set (e.g., TCGA). This threshold aligns with clinical priorities, favouring the detection of positive cases.

To facilitate interpretability
a. Raw probabilities (p ε [0,1]) were transformed into a scaled confidence score (s ε [0,100]) using a custom function which centers the probability distribution around the sensitivity-derived threshold.
b. This normalization maps the threshold to a pivot point (e.g., 50) in the scaled space, providing a more intuitive representation of relative risk or confidence.

To quantify prediction reliability and inter-model agreement:
a. For each test sample, raw and scaled probabilities were aggregated across all selected models.
b. The following metrics were computed across the ensemble distribution: Mean prediction, Standard deviation, 95% confidence interval (CI).

Uncertainty is computed as the width of the 95% CI in the scaled probability space.
Uncertainty = CIᵤₚₚₑᵣ – CI_{lower}.

Finally, Confidence is calculated either through Uncertainty.

This structured approach not only improves predictive robustness via model ensembling but also provides a principled way to interpret prediction certainty, empowering downstream decision-making with calibrated and transparent confidence estimates.

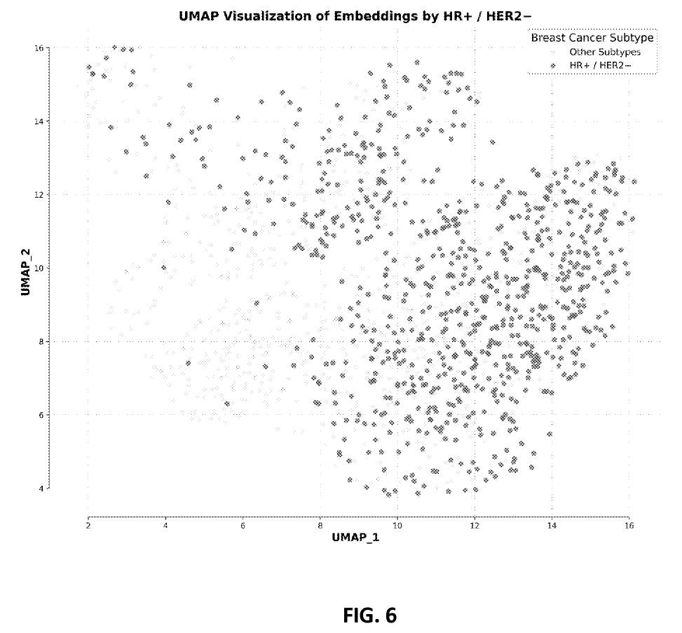

FIG. 6 illustrates a visualization of TRINITY AI's Learned Multi-Modal Embeddings by HR+/HER2-. Two-dimensional UMAP projections of sample-level embeddings generated by TRINITY AI illustrate the semantic structure of the shared representation space. Cases of the indicated subtype are shown in blue, and all others in gray. The consistent enrichment and partial clustering of each subtype demonstrate that TRINITY AI embeddings encode biologically meaningful, clinically relevant variation across modalities. The absence of batch effects or random dispersion confirms the robustness and generalizability of the learned representation for downstream predictive tasks.

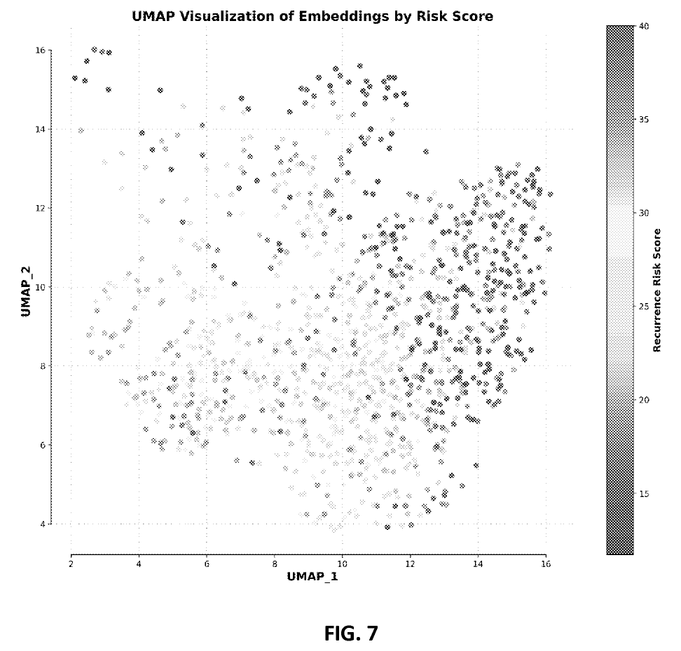

FIG. 7 illustrates a UMAP Visualization of TRINITY AI Embeddings by recurrence risk scores. Two-dimensional UMAP projection of multi-modal embeddings colored by recurrence risk scores. The embeddings demonstrate coherent organization aligned with clinical risk scores, indicating that TRINITY AI captures biologically relevant, structure.

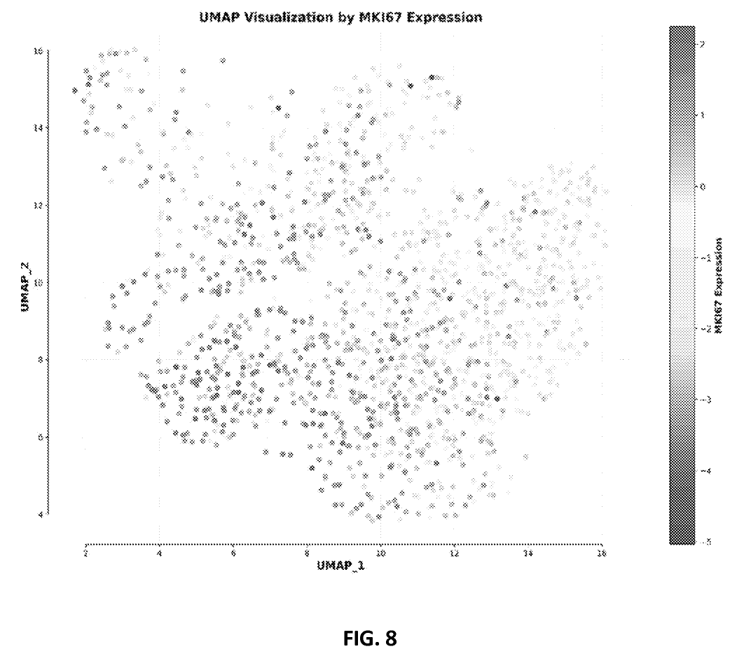

FIG. 8 illustrates a UMAP Visualization by MKI67 Expression. UMAP embedding colored by MKI67, a proliferation marker. TRINITY AI embeddings exhibit localized high MKI67 expression regions, reflecting biologically meaningful variance in tumour proliferation rates.

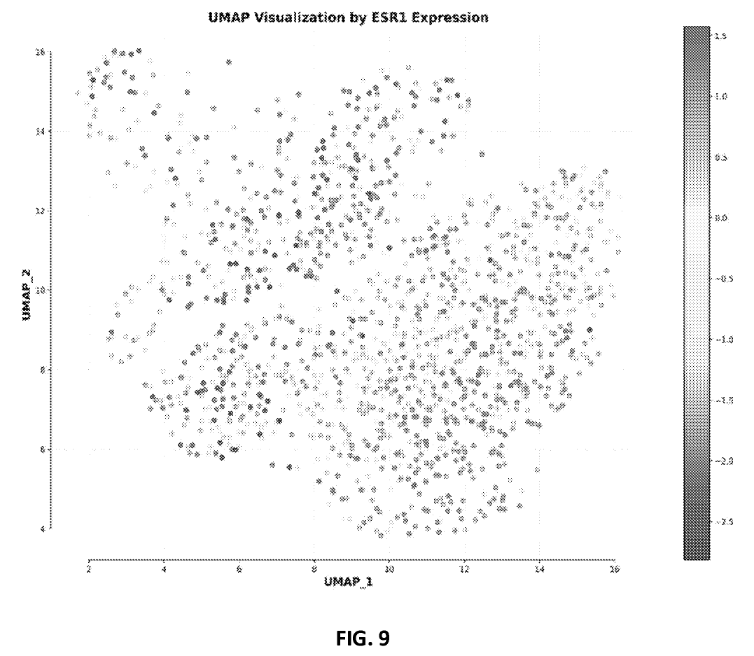

FIG. 9 illustrates a UMAP Visualization by ESR1 Expression. UMAP plot colored by ESR1 (Estrogen receptor) expression. A clear gradient structure is observed, with distinct regions enriched in high or low ESR1 expression. This alignment reinforces the model's capacity to encode hormone receptor signaling patterns within its embedding space.

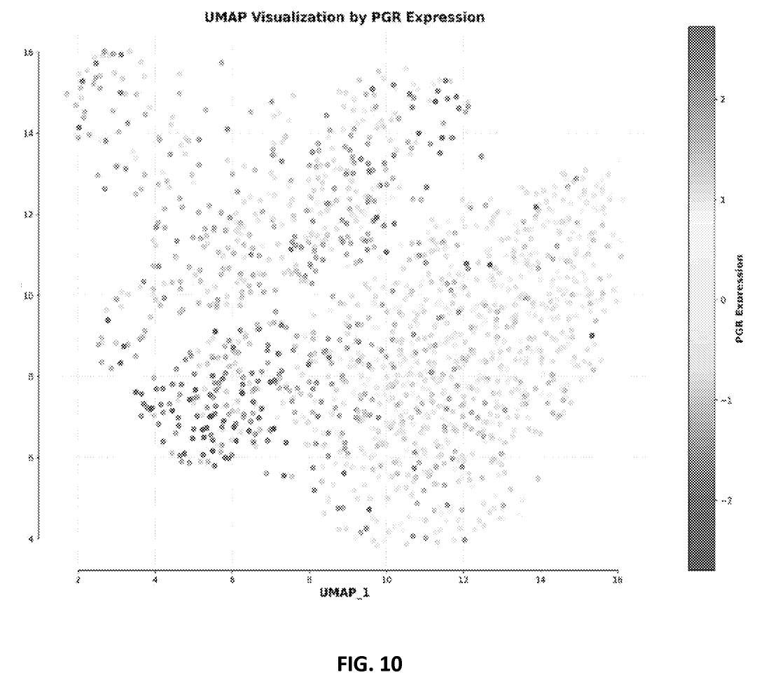

FIG. 10 illustrates a UMAP Visualization by PGR Expression. UMAP plot colored by PGR (progesterone receptor) gene expression. The expression levels form a structured distribution across the embedding space, consistent with TRINITY AI's encoding of hormone receptor status relevant to breast cancer biology.

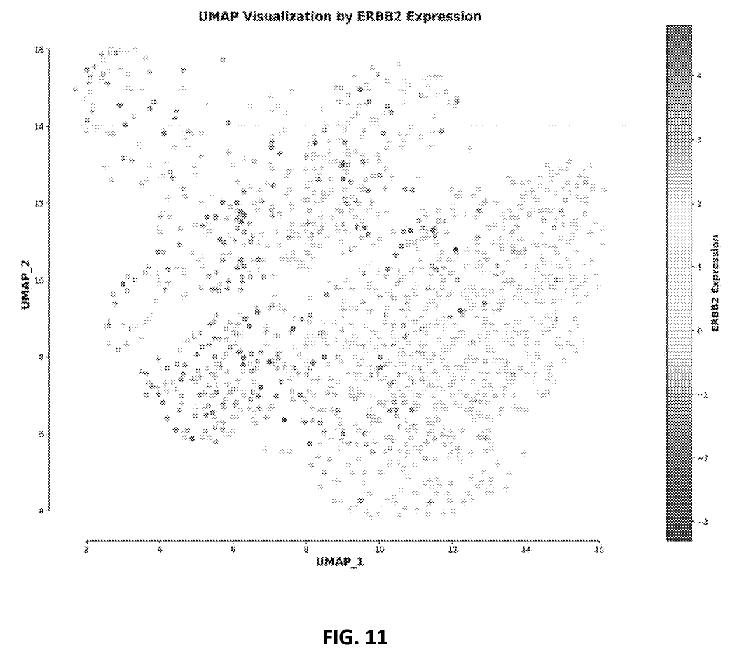

FIG. 11 illustrates a UMAP Visualization by ERBB2 Expression. Sample embeddings colored by normalized ERBB2 gene expression. Localized gradients and clusters enriched in ERBB2 levels suggest that TRINITY AI’s latent space is sensitive to HER2 pathway activity.

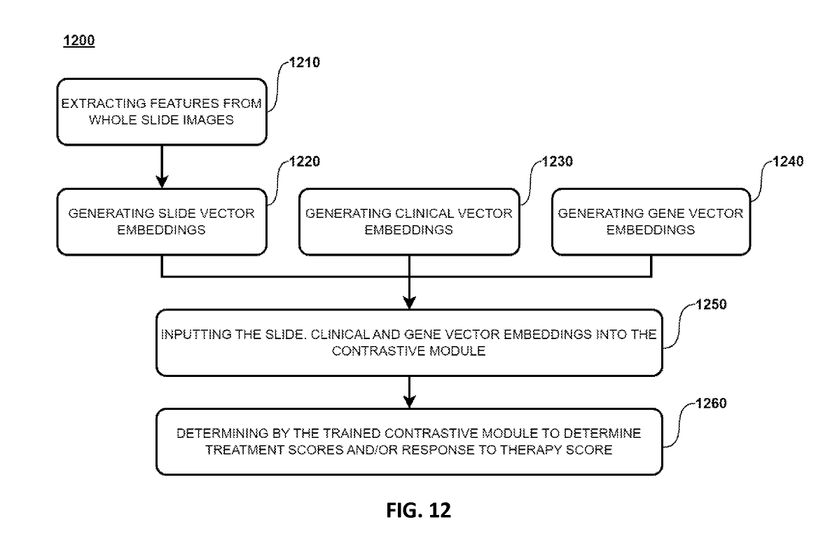

FIG. 12 is a diagram illustrating an exemplary process 1200 that may perform processing in some embodiments. In some embodiments, the system performs a multi-modal artificial neural network-based method and system for predicting patient response and biomarkers for precision oncology from histopathological whole slide images (WSIs) only. Utilizing an artificial neural network (ANN) with a feature extraction module, image to gene expression prediction, encoders and contrastive learning-based methods the system processes WSIs to determine treatment scores from predicted profiles, forecasting responses to therapies like chemotherapy, targeted drugs, and immunotherapy across cancers (e.g., breast). Applications include predicting recurrence risk, treatment efficacy (e.g., PARP inhibitors, checkpoint inhibitors), biomarker status, patient stratification, clinical trial design, drug target identification, and therapeutic combination efficiency.

In step 1210, the system receives whole slide images (WSI) each comprising an image file of multiple pixels. The system extracts image features from the received WSI images using one or more feature extraction architectures comprising one or more of a convolutional neural network, a vision transformer, or other foundation model.

In step 1230, the system generates clinical vector embeddings by inputting numerical clinical data into a clinical data encoder.

In step 1220, the system generates slide vector embeddings by inputting the extracted image features via a slide encoder.

In step 1240, the system generates vector embeddings by inputting into a gene encoder scout gene expressions.

In step 1250, the system inputs into a contrastive module comprising a machine learning model, the generated clinical vector embeddings, the slide vector embeddings and the gene vector embedding.

In step 1260, the system determines by the trained contrastive module a treatment score and/or response to therapy and then provide the score for display via a user interface.

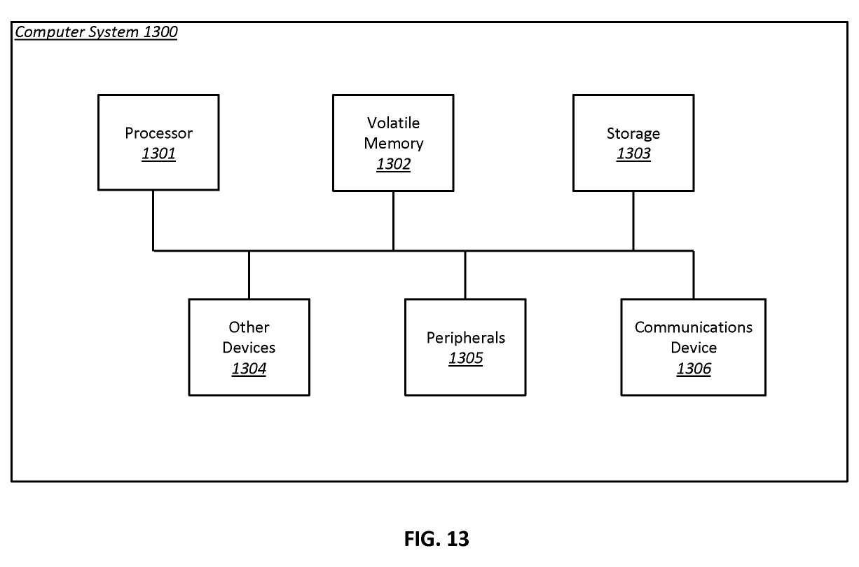

FIG. 13 is a diagram illustrating an exemplary computer system 1300 that may perform processing in some embodiments. Processor 1301 may perform computing functions such as running computer programs. The volatile memory 1302 may provide temporary storage of data for the processor 1301. RAM is one kind of volatile memory. Volatile memory typically requires power to maintain its stored information. Storage 1303 provides computer storage for data, instructions, and/or arbitrary information. Non-volatile memory, which can preserve data even when not powered and including disks and flash memory, is an example of storage. Storage 1303 may be organized as a file system, database, or in other ways. Data, instructions, and information may be loaded from storage 1303 into volatile memory 1302 for processing by the processor 1301.

The computer system 1300 may include peripherals 1305. Peripherals 1305 may include input peripherals such as a touch screen, keyboard, pen, mouse, trackball, video camera, microphone, and other input devices. Peripherals 1305 may also include output devices such as a display. Communications device 1306 may connect the computer system 1300 to an external medium. For example, communications device 1306 may take the form of a network adapter that provides communications to a network. The computer system 1300 may also include a variety of other devices 1304. The various components of the computer system 1300 may be connected by a connection medium such as a bus, crossbar, or network.

It will be appreciated that the present disclosure may include any one and up to all of the following examples.

Example 1. A computer-implemented method comprising: receiving whole slide images (WSI) each comprising an image file of multiple pixels; extracting image features from the received WSI images using one or more feature extraction architectures comprising one or more of a convolutional neural network, a vision transformer, or other foundation model; generating clinical vector embeddings by inputting numerical clinical data into a clinical data encoder; generating slide vector embeddings by inputting the extracted image features via a slide encoder; generating gene vector embeddings by inputting into a gene encoder scout gene expressions; inputting into a contrastive module comprising a machine learning model, the generated clinical vector embeddings, the slide vector embeddings and the gene vector embeddings; determining by the contrastive module a treatment score and/or a response to therapy; an providing for display, via a user interface an indication of the treatment score and/or the response to therapy.

Example 2. The computer-implemented method of Example 1, further comprising: receiving clinical data; and generating the numerical clinical data via a numerical data encoder.

Example 3. The computer-implemented method of any one of Examples 1-2, the trained machine learning model utilizes a shared representation space using a contrastive loss function to evaluate the input the generated clinical vector embeddings, the slide vector embeddings and the gene vector embeddings.

Example 4. The computer-implemented method of any one of Examples 1-3, further comprising the operations of: inputting into bulk RNA-sequence data into a transcript module and generating gene expression data.

Example 5. The computer-implemented method of any one of Examples 1-4 wherein the transcript module comprises a machine learning model trained by: (a) providing a training dataset of paired histopathological images and corresponding bulk RNA-seq data from the same tissue samples; (b) preprocessing the histopathological images through normalization, quality control, and patch extraction; (c) preprocessing the RNA-seq data through normalization and batch correction; and (d) training the transcript machine learning model to minimize the difference between predicted gene expression values and actual gene expression values measured by bulk RNA-seq.

Example 6. The computer-implemented method of any one of Examples 1-5, further comprising the operations of: inputting gene expression data into a scout module and generating the scout gene expressions.

Example 7. The computer-implemented method of any one of Examples 1-6, wherein the scout module is configured to a) receive a comprehensive gene expression profile comprising thousands of predicted genes from the transcript module; b) implement multiple selection approaches including differential gene expression analysis and genetic model and process-based selection; c) identify a reduced subset of genes with optimal predictive value for specific clinical outcomes; and d) output a refined gene expression profile containing approximately subset of genes with heightened relevance to the target condition.

Example 8. The computer-implemented method of any one of Examples 1-7, further comprising: extracting pixels in the WSI images of the real tissue regions of interest by excluding artefacts, staining/sectioning errors, blurry and unwanted biological substances such as fatty tissue, small objects, and white background fatty regions.

Example 9. The computer-implemented method of any one of Examples 1-8, further comprising: segmenting each of the WSI images into discrete tiles of pixels; and inputting the extracted tiles into a feature extractor module that generates histopathological features represented as the extracted image features.

Example 10. The computer-implemented method of any one of Examples 1-9, further comprising: downscaling each of the WSI images to match a resolution of a mask.

Example 11. A system comprising one or more processors configured to perform the operations of: receiving whole slide images (WSI) each comprising an image file of multiple pixels; extracting image features from the received WSI images using one or more feature extraction architectures comprising one or more of a convolutional neural network, a vision transformer, or other foundation model; generating clinical vector embeddings by inputting numerical clinical data into a clinical data encoder; generating slide vector embeddings by inputting the extracted image features via a slide encoder; generating gene vector embeddings by inputting into a gene encoder scout gene expressions; inputting into a contrastive module comprising a machine learning model, the generated clinical vector embeddings, the slide vector embeddings and the gene vector embeddings; determining by the contrastive module a treatment score and/or a response to therapy; an providing for display the determined treatment score and/or the response to therapy.

Example 12. The system of Example 11, further comprising: receiving clinical data; and generating the numerical clinical data via a numerical data encoder.

Example 13. The system of any one of Examples 11-12, the trained machine learning model utilizes a shared representation space using a contrastive loss function to evaluate the input the generated clinical vector embeddings, the slide vector embeddings and the gene vector embeddings.

Example 14. The system of any one of Examples 11-13, further comprising the operations of: inputting into bulk RNA-sequence data into a transcript module and generating gene expression data.

Example 15. The system of any one of Examples 11-14, wherein the transcript module comprises a machine learning model trained by: (a) providing a training dataset of paired histopathological images and corresponding bulk RNA-seq data from the same tissue samples; (b) preprocessing the histopathological images through normalization, quality control, and patch extraction; (c) preprocessing the RNA-seq data through normalization and batch correction; and (d) training the transcript machine learning model to minimize the difference between predicted gene expression values and actual gene expression values measured by bulk RNA-seq.

Example 16. The system of any one of Examples 11-15, further comprising the operations of: inputting gene expression data into a scout module and generating the scout gene expressions.

Example 17. The system of any one of Examples 11-16, wherein the scout module is configured to a) receive a comprehensive gene expression profile comprising thousands of predicted genes from the transcript module; b) implement multiple selection approaches including differential gene expression analysis and genetic model and process-based selection; c) identify a reduced subset of genes with optimal predictive value for specific clinical outcomes; and d) output a refined gene expression profile containing approximately subset of genes with heightened relevance to the target condition.

Example 18. The system of any one of Examples 11-17, further comprising the operations of: extracting pixels in the WSI images of the real tissue regions of interest by excluding artefacts, staining/sectioning errors, blurry and unwanted biological substances such as fatty tissue, small objects, and white background fatty regions.

Example 19. The system of any one of Examples 11-18, further comprising the operations of: segmenting each of the WSI images into discrete tiles of pixels; inputting the extracted tiles into a feature extractor module that generates histopathological features represented as the extracted image features.

Example 20. The system of c any one of Examples 11-19, further comprising the operations of: downscaling each of the WSI images to match a resolution of a mask.

Some portions of the preceding detailed descriptions have been presented in terms of processes, functions and/or symbolic representations of operations on data bits within a computer memory. These model and processes and/or equation descriptions and representations are the ways used by those skilled in the data processing arts to most effectively convey the substance of their work to others skilled in the art. A model and process is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “identifying” or “determining” or “executing” or “performing” or “collecting” or “creating” or “sending” or the like, refer to the action and processes of a computer system, or similar electronic computing device, that manipulates and transforms data represented as physical (electronic) quantities within the computer system’s registers and memories into other data similarly represented as physical quantities within the computer system memories or registers or other such information storage devices.

The present disclosure also relates to an apparatus for performing the operations herein. This apparatus may be specially constructed for the intended purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

Various general-purpose systems may be used with programs in accordance with the teachings herein, or it may prove convenient to construct a more specialized apparatus to perform the method. The structure for a variety of these systems will appear as set forth in the description above. In addition, the present disclosure is not described with reference to any programming language. It will be appreciated that a variety of programming languages may be used to implement the teachings of the disclosure as described herein.

The present disclosure may be provided as a computer program product, or software, that may include a machine-readable medium having stored thereon instructions, which may be used to program a computer system (or other electronic devices) to perform a process according to the present disclosure. A machine-readable medium includes any mechanism for storing information in a form readable by a machine (e.g., a computer). For example, a machine-readable (e.g., computer-readable) medium includes a machine (e.g., a computer) readable storage medium such as a read only memory (“ROM”), random access memory (“RAM”), magnetic disk storage media, optical storage media, flash memory devices, etc.

In the foregoing disclosure, implementations of the disclosure have been described with reference to specific example implementations thereof. It will be evident that various modifications may be made thereto without departing from the broader spirit and scope of implementations of the disclosure as set forth in the following claims. The disclosure and drawings are, accordingly, to be regarded in an illustrative sense rather than a restrictive sense.

What is claimed is:

1. The computer implemented method comprising:
receiving whole slide images (WSI) each comprising an image file of multiple pixels;
extracting image features from the received WSI images using one or more feature extraction architectures comprising one or more of a convolutional neural network, a vision transformer, or other foundation model;
generating clinical vector embeddings by inputting numerical clinical data into a clinical data encoder;
generating slide vector embeddings by inputting the extracted image features via a slide encoder;
generating gene vector embeddings by inputting scout gene expressions into a gene encoder;
inputting into a contrastive module comprising a machine learning model, the generated clinical vector embeddings, the slide vector embeddings and the gene vector embeddings, and determining a treatment score and/or a response to therapy score; and
providing for display an indication of the determined score.

2. The computer-implemented method of claim 1, further comprising:
receiving clinical data; and
generating the numerical clinical data via a numerical data encoder.

3. The computer-implemented method of claim 1, the trained machine learning model utilizes a shared representation space using a contrastive loss function to evaluate the input the generated clinical vector embeddings, the slide vector embeddings and the gene vector embeddings.

4. The computer-implemented method of claim 1, further comprising the operations of:
inputting into bulk RNA-sequence data into a transcript module and generating gene expression data.

5. The computer-implemented method of claim 4, wherein the transcript module comprises a machine learning model trained by:
(a) providing a training dataset of paired histopathological images and corresponding bulk RNA-seq data from the same tissue samples;
(b) preprocessing the histopathological images through, quality control, and patch extraction;
(c) preprocessing the RNA-seq data through normalization and batch correction; and
(d) training the transcript machine learning model to minimize the difference between predicted gene expression values and actual gene expression values measured by bulk RNA-seq.

6. The computer-implemented method of claim 4, further comprising the operations of: inputting gene expression data into a scout module and generating the scout gene expressions.

7. The computer-implemented method of claim 6, wherein the scout module is configured to a) receive a comprehensive gene expression profile comprising thousands of predicted genes from the transcript module; b) implement multiple selection approaches including differential gene expression analysis and genetic model and process-based selection; c) identify a reduced subset of genes with optimal predictive value for specific clinical outcomes; and d) output a refined gene expression profile containing approximately subset of genes with heightened relevance to the target condition.

8. The computer-implemented method of claim, further comprising:
extracting pixels in the WSI images of the real tissue regions of interest by excluding artefacts, staining/sectioning errors, blurry and unwanted biological substances such as fatty tissue, small objects, and white background fatty regions.

9. The computer-implemented method of claim 1, further comprising:
segmenting each of the WSI images into discrete tiles of pixels; and
inputting the extracted tiles into a feature extractor module that generates histopathological features represented as the extracted image features.

10. The computer-implemented method of claim 1, further comprising:
downscaling each of the WSI images to match a resolution of a mask.

11. A system comprising one or more processors configured to perform the operations of:
receiving whole slide images (WSI) each comprising an image file of multiple pixels;
extracting image features from the received WSI images using one or more feature extraction architectures comprising one or more of a convolutional neural network, a vision transformer, or other foundation model;
generating clinical vector embeddings by inputting numerical clinical data into a clinical data encoder;
generating slide vector embeddings by inputting the extracted image features via a slide encoder;
generating gene vector embeddings by inputting into a gene encoder scout gene expressions;
inputting into a contrastive module comprising a machine learning model, the generated clinical vector embeddings, the slide vector embeddings and the gene vector embeddings, and determining a treatment score and/or a response to therapy score; and
providing for display an indication of the determined score.

12. The system of claim 11, further comprising:
receiving clinical data; and
generating the numerical clinical data via a numerical data encoder
receiving numerical clinical data.

13. The system of claim 11, the trained machine learning model utilizes a shared representation space using a contrastive loss function to evaluate the input the generated clinical vector embeddings, the slide vector embeddings and the gene vector embeddings.

14. The system of claim 11, further comprising the operations of:
inputting into bulk RNA-sequence data into a transcript module and generating gene expression data.

15. The system of claim 14, wherein the transcript module comprises a machine learning model trained by:
(a) providing a training dataset of paired histopathological images and corresponding bulk RNA-seq data from the same tissue samples;
(b) preprocessing the histopathological images through normalization, quality control, and patch extraction;
(c) preprocessing the RNA-seq data through normalization and batch correction; and
(d) training the transcript machine learning model to minimize the difference between predicted gene expression values and actual gene expression values measured by bulk RNA-seq.

16. The system of claim 14, further comprising the operations of: inputting gene expression data into a scout module and generating the scout gene expressions.

17. The system of claim 16, wherein the scout module is configured to a) receive a comprehensive gene expression profile comprising thousands of predicted genes from the transcript module; b) implement multiple selection approaches including differential gene expression analysis and genetic model and process-based selection; c) identify a reduced subset of genes with optimal predictive value for specific clinical outcomes; and d) output a refined gene expression profile containing approximately subset of genes with heightened relevance to the target condition.

18. The system of claim 11, further comprising the operations of:
extracting pixels in the WSI images of the real tissue regions of interest by excluding artefacts, staining/sectioning errors, blurry and unwanted biological substances such as fatty tissue, small objects, and white background fatty regions.

19. The system of claim 11, further comprising the operations of:
segmenting each of the WSI images into discrete tiles of pixels; and
inputting the extracted tiles into a feature extractor module that generates histopathological features represented as the extracted image features.

20. The system of claim 11, further comprising the operations of:
downscaling each of the WSI images to match a resolution of a mask.
